(19) United States
(12) Patent Application Publication
(10) Pub. No.: US 2009/0234139 A1
(43) Pub. Date: Sep. 17, 2009

(54) Novel Process

(75) Inventors: Lovro Selic, Celje (SI); Gregor Vilfan, Ljubljana (SI); Uros Urleb, Ljubljana (SI)

Correspondence Address: SANDOZ INC, 506 CARNEFIE CENTER, PRINCETON, NJ, 08540, US

(73) Assignee: LEK PHARMACEUTICALS, D.D., LJUBLJANA (SR)

(21) Appl. No.: 11/992,212

(22) PCT Filed: Sep. 19, 2006
(86) PCT No.: PCT/EP2006/009101

§ 371 Date: Mar. 18, 2008

(30) Foreign Application Priority Data
Sep. 21, 2005 (SI) P200500269

(51) Int. Cl. C07D 205/04

(52) U.S. Cl. 548/952

(57) ABSTRACT

A novel process for the preparation of 1′-hydroxy-2′-substituted cyclohexyl azetidin-2-one compound of formula 2, which is important intermediate in the synthesis of trinems, is described by epoxide ring opening of (3S,4R)-3-[(1R)-1-(tert-butyldimethylsilyloxy)ethyl]-4-[(1′R,2′S,3′R)-1′,2′-epoxycyclohexan-3′-yl]azetidin-2-one of formula 1 with the nucleophile compound of formula RYH, where nucleophile may act as solvent itself if the nucleophile is in the liquid form, in a suitable solvent and in the presence of a suitable catalyst from the group of salt of trifluoromethane sulfonic acid, preferably ytterbium (III) trifluoromethanesulfonate, stannous (II) trifluoromethanesulfonate or dysprosium (III) trifluoromethanesulfonate, under a) ultrasonic irradiation or b) under microwave irradiation of the reaction mixture following by isolation and purification of the desired compound. A variant of the novel process describes ring epoxide opening of the starting compound of formula 1 with the compound of formula NH₄X in a suitable solvent under a) microwave irradiation or b) without microwave irradiation to obtain the desired compound of formula 2. Instead of compound of formula 1 a compound of formula 6 may be used as starting compound where any other suitable hydroxy protecting group known in the art may be used in the formula of the starting compound.

The present invention belongs to the field of heterocyclic chemistry and relates to a novel process for the preparation of 1′-hydroxy-2′-substituted cyclohexyl azetidin-2-ones, which are important intermediates in the synthesis of trinems, a new family of totally synthetic β-lactam antibiotics.

C. Marchioro et al., J. Chem. Soc., Perkin Trans. 1, 1997, 463-468 describes epoxide ring opening of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (hereinafter referred to compound 1), starting from the commercially available derivative of 4-acetoxyazetidinone, in methanol using p-toluene sulfonic acid monohydrate as a catalyst resulting (3S)-3-((R)-1-(tert-butyldimethylsilyloxy)ethyl)-4-((1R,2S,3S)-2-hydroxy-3-methoxycyclohexyl)azetidin-2-one (hereinafter referred to compound 2a) in 65% yield. The said compound is an important intermediate in the synthesis of trinems.

D. Andreotti et al., Bioorg. & Med. Chem. Leff., Vo. 6, No. 16, pp. 2019-2024, 1996 describes the preparation of (3S)-3-((R)-1-(tert-butyldimethylsilyloxy)ethyl)-4-((1R,2S,3S)-3-(2-fluoroethoxy)-2-hydroxycyclohexyl)azetidin-2-one (hereinafter referred to compound 2f) from the above compound 1 in 2-fluoroethanol as solvent in the absence of catalyst in 21% yield and the preparation of 3-((1S,2S,3R)-3-((3S)-3-((R)-1-(tert-butyldimethylsilyloxy)ethyl)-4-oxoazetidin-yl)-2-hydroxycyclohexyl) propanenitrile (hereinafter referred to compound 2h) from the above compound 1 in 3-hydroxypropanenitrile as solvent in very low yield, which increased to 20% by using cerium ammonium (IV) nitrate instead of p-toluenesulfonic acid as acidic catalyst.

Surprisingly, the present inventors have found a novel process for the preparation of various 1′-hydroxy-2′-substituted cyclohexyl azetidin-2-one of general formula 2

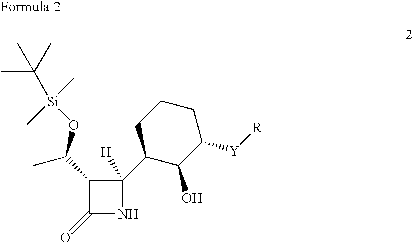

wherein

### Y is O, S or NH;

R is hydrogen, or selected from a) phenyl group optionally substituted by one or two substituents each selected from nitro, halo, C₁-C₄ alkyl, C₁-C₄ alkoxy, hydroxy, trifluoromethyl and cyano, b) a 1- or 2-naphthyl group, and c) oxyranyl, aziridinyl, pirolyl, imidazolyl, pyrazolyl, furanyl, thiophenyl, thiazolyl, isothiazolyl, triazolyl, tetrazolyl, oxazolyl, isooxazolyl, azepinyl, pyridinyl, pyrimidinyl, pyrazinyl, pyridazinyl, triazinyl, pyranyl and their hydrogenated analogs, unsubstituted or optionally substituted by one or two substituents each selected from nitro, nitroso, amino, imino, sulfonyl, chlorosulfonyl, halo, C₁-C₄ alkyl, C₁-C₄ alkoxy, C₁-C₄ alkoxycarbonyl, C₁-C₄ alkylthio, C₁-C₄ alkylthiocarbonyl, hydroxy, trifluoromethyl, carboxyaldehyde or cyano group; or R₁(CHₘ)ₙ— group where n is an integer from 1 to 11, m is an integer from 1 to 2 and R₁ is a) hydrogen, halo, hydroxy, ciano, C₁-C₄ alkoxy, C₁-C₄ alkoxycarbonyl, nitro, nitroso, amino, imino, sulfonyl, chlorosulfonyl, C₁-C₄ alkylthio, C₁-C₄ alkylthiocarbonyl; or b) C₁-C₆ alkyl, C₂-C₆ alkenyl, C₃-C₇ cycloalkyl, optionally substituted by one or two substituents each selected from halo, hydroxy, ciano, carboxyaldehyde, C₁-C₄ alkoxy, C₁-C₄ alkoxycarbonyl, nitro, nitroso, amino, imino, sulfonil, chlorosulfonyl, C₁-C₄ alkylthio, C₁-C₄ alkylthiocarbonyl group;
in moderate to excellent yield by epoxide ring opening of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound of formula 1)

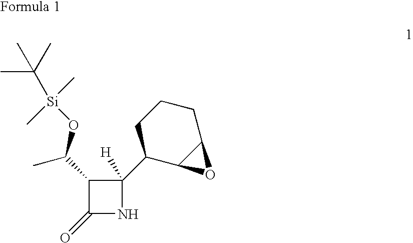

with the nucleophile compound of general formula 4

RYH Formula 4

wherein Y and R are as defined above
where nucleophile if in a form of liquid can act as a solvent itself, in a suitable solvent and in the presence of a suitable catalyst under a) ultrasonic irradiation or b) microwave irradiation of the reaction mixture, as illustrated by the following scheme 1:

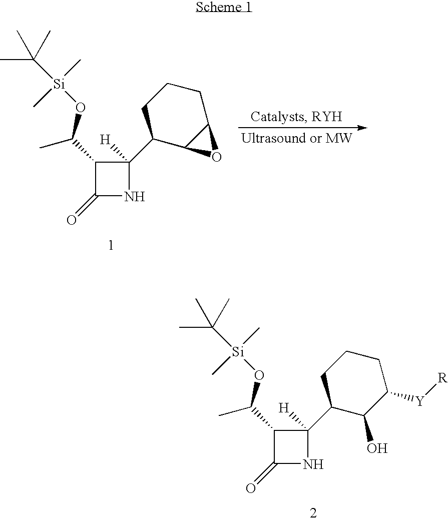

(3S,4R)-3-[(1R)-1-(tert-butyldimethylsilyloxy)ethyl]-4-[(1′R, 2′S,3′R)-1′,2′-epoxycyclohexan-3′-yl]azetidin-2-one (compound of formula 1) as starting compound may be prepared according to the description in article of C. Marchioro et al., J. Chem. Soc., Perkin Trans. 1, 1997, 463-468.

A suitable solvent medium may be nucleophile itself if in the form of liquid or may comprise water, C₁-C₆ alcohol, e.g. methanol, ethanol, n-propanol, iso-propanol, n-butanol, sec-butanol, iso-butanol, tert-butanol, N,N-dimethylformamide, N,N-dimethylacetamide, acetonitrile, tetrahydrofuran (THF), dioxane, 1-methylpyrrolidinone, 1,3-dimethyl-3,4,5,6-tetrahydro-2(1H)-pyrimidone, dichloromethane, dimethylsulfoxide, liquid lower C₁-C₄ alkyl acetate, e.g. ethyl acetate, aromatic hydrocarbon, e.g. toluene, benzene, or mixtures thereof.

A suitable catalyst comprise Lewis acid, preferably salt of trifluoromethanesulfonic acid, more preferably ytterbium (III) triflate, stannous (II) triflate and dysprosium (III) triflate.

Reaction may be performed under ultrasonic irradiation or alternatively under microwave irradiation of the reaction mixture.

In a preferred embodiment the reaction may be performed in an ultrasonic bath without heating. However, the temperature of a reaction mixture rises spontaneously from about room temperature to about 60° C. The reaction may be performed in the frequency range from 20 to 80 kHz, preferably from 30 to 50 kHz.

During the reaction process the suitable catalyst may be added to the reaction mixture which is immersed in the ultrasonic bath, e.g. for about 1 hour. If reaction is not completed another portion of the catalyst is added to the reaction mixture and placed for another period, e.g. for about 1 hour, in the ultrasonic bath. The process may be repeated until conversion is completed. Generally, two to four times of adding the catalyst in immersing the reaction mixture in the ultrasonic bath is necessary to complete the conversion. Larger portions of catalyst and/or longer reaction time instead of multiple addition do not lead to total conversion of starting compound.

After the completing of the process the reaction mixture is evaporated and the resulted product may be purified by the common technique, e.g. by extraction and/or crystallisation or precipitation by means of organic non-polar solvent, e.g. pentane, hexane, heptane, di-C₁-C₄ alkyl ethers, ethyl acetate, and petroleum ether.

In a preferred embodiment the compound of formula 1 is converted with the suitable C₁-C₄ alcohol, e.g. methanol, ethanol, n-propanol, iso-propanol, n-butanol, iso-butanol, as nucleophilic compound, and several additions, e.g. four additions, of ytterbium (III) triflate as the catalyst to the reaction mixture immersed in the ultrasonic bath.

On the basis of the ultrasonic irradiation of the reaction mixture a significally improved yields of 1′-hydroxy-2′-substituted cyclohexyl azetidin-2-one of general formula 2 in comparison with the prior art processes may be obtained.

In another embodiment instead of using ultrasonic irradiation of the above described reaction mixture, the suitable catalyst may be added to the said reaction mixture under microwave irradiation.

Reaction may be performed under microwave irradiation from about 30 seconds to several hours, preferable for about one hour. Temperature of the reaction may be from room temperature to about 250° C., preferably from about 60 to about 80° C., depending on the solvent used. After completing of the reaction process the resulted product may be precipitated from the reaction mixture by addition of water and dried the precipitate to obtain the desired 1′-hydroxy-2′-substituted cyclohexyl azetidin-2-one derivative of the general formula 2.

In a further aspect of the invention various 1′-hydroxy-2′-substituted cyclohexyl azetidin-2-one of general formula 3

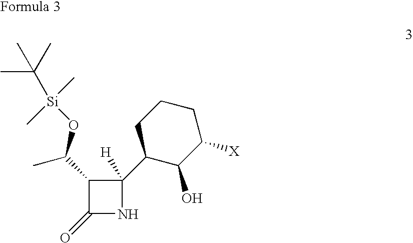

wherein X is halogen, e.g. chlorine, bromine and fluorine, thiocyanate, isothiocyanate, hydroxy, sulphate, nitro, sulfide, sulfite, carbonate or acetate group, may be prepared by expoxide ring opening of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound of formula 1) with the compound of general formula 5

NH₄X formula 5

wherein X is as defined above, in a suitable solvent under a) microwave irradiation or alternatively b) without microwave irradiation, as illustrated by the following scheme 2:

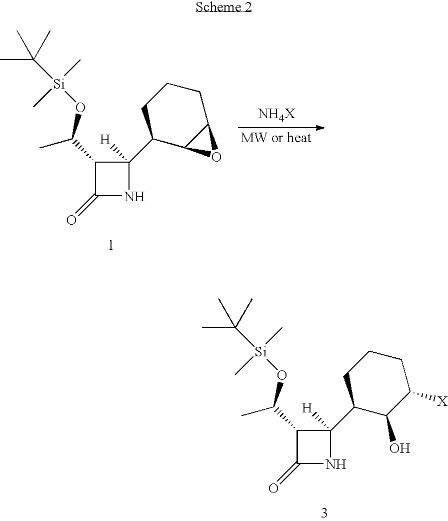

A suitable solvent medium comprise the polar solvent used in above described process, e.g. water, C₁-C₄ alcohol, e.g. methanol or ethanol, acetonitrile, ethyl acetate etc.

Reaction may be performed under microwave irradiation by stirring the reaction mixture for several hours from about room temperature to about 250° C., preferably from about 60 to 150° C., more preferably from about 80 to 130° C., depending on the solvent used, then concentrated the reaction mixture and the concentrate dissolved in a suitable solvent, e.g. ethyl acetate; the obtained solution washed with a base comprising an alkaline carbonate or earth alkaline carbonate, e.g. sodium hydrogen carbonate, sodium carbonate, potassium carbonate; dried, e.g. over MgSO₄ or Na₂SO₄, and finally purified by the known techniques, e.g. by flash chromatography. Reaction according to the invention may be performed for about one to about 24 hours, preferably for about 3 hours. Reaction may be performed also without microwave irradiation, at reflux temperature for about 1 day to about 2 days, however, with considerable lower yield.

Compound of general formula 3 is useful intermediate for preparing trinems.

All the above described processes may be performed according to a further aspect of the invention, where instead of using compound of formula 1a compound of the following formula 6

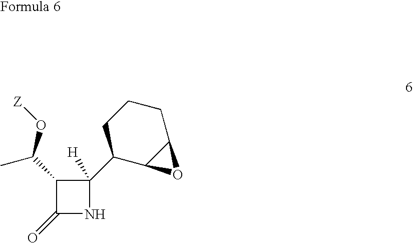

may be used as starting compound, wherein
Z is another suitable hydroxy protecting group. Thus the group —OZ may be an ether or an acyloxy group known in the art, for example as described in the textbook T. W. Greene and P. G. M. Wuts, Protective Groups in Organic Synthesis, 3^{rd} Ed., 1999, under the heading “Protection for the Hydroxyl Group, Ethers, 23-244, Esters, 149-210 or as summarized in Reaction Chart 1. Protection for Hydroxyl group: Ethers 712-715 and Reaction Chart 2. Protection for Hydroxyl Group: Esters 712-715”.

Examples of particularly suitable ethers include those in which Z is a hydrocarbosilyl group such as tri(C₁-C₆)alkylsilyl, e.g. trimethyl silyl. In the above starting compound of formula 1a particularly convenient hydroxy protection group is tert-butyldimethylsilyl.

The invention is illustrated by the following examples. All temperatures are given in degree Celsius and are uncorrected.

### EXAMPLE 1

General method for the preparation of compound of formula 2 using ultrasonic irradiation. Experiments are performed in a Cole-Parmer 8890 ultrasonic bath.

### Preparation of (3S)-3-((R)-1-(tert-butyldimethylsilyloxy)ethyl)-4-((1R,2S,3S)-2-hydroxy-3-methoxycyclohexyl)azetidin-2-one (compound 2a)

To a solution of 372 mg (1.14 mmol) of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound 1) in methanol (3 ml), a portion (21 mg, 3 mol %) of ytterbium (III) trifluoromethane sulfonate is added, a solution is immersed for 60 min into ultrasonic bath at frequency output 42 kHz. Then another portion of ytterbium (III) trifluoromethane sulfonate is added and immersed for 60 min into ultrasonic bath. This is repeated two more times (4× cumulative). Reaction mixture is concentrated in vacuo, oily residue is dissolved in ethyl acetate and the obtained solution washed with saturated ammonium chloride solution, dried over MgSO₄ and evaporated. The obtained white substance is crystallized from hexane to give 343 mg (84%) of the title compound.

m.p. 146-148° C. (from hexane)

¹H NMR (300 MHz, CDCl₃): δ 0.08 (s, 6H), 0.88 (s, 9H), 1.27 (d, J=6.2 Hz, 3H), 1.25-1.93 (m, 8H), 2.95 (m, 1H), 3.33 (s, 3H), 3.34 (m, 1H), 3.64 (dd, J=2.1, 6.8 Hz, 1H) 3.81 (m, 1H), 4.17 (m, 1H), 6.02 (bs, 1H).

¹³C NMR (300 MHz, CDCl₃): δ −4.6, −4.3, 18.0, 19.1, 21.5, 22.7, 23.7, 25.8 (3), 39.9, 53.7, 56.3, 61.8, 66.3, 69.9, 79.1, 169.0

According to the above general method the following compounds using ultrasonic irradiation are prepared:

### EXAMPLE 2

### Preparation of (3S)-3-((R)-1-(tert-butyldimethylsilyloxy)ethyl)-4-((1R,2S,3S)-2-hydroxy-3-ethoxycyclohexyl)azetidin-2-one (compound 2b)

Instead of using methanol as in example 1, ethanol is used as a nucleophilic compound resulting the title compound in 75% yield. 4×3 mol % Yb(OTf)₃ is used as a catalyst.

m.p. 136-138° C. (from hexane)

MS, (M+H)⁺=372 m/z.

IR (KBr): 3447, 3281, 2934, 2857, 1760, 1714, 1256, 1142, 1106, 1085, 962, 836, 776 cm⁻¹.

¹H NMR (300 MHz, DMSO-d₆): δ 0.09 (s, 6H), 0.89 (s, 9H), 1.18 (t, J=7.0 Hz, 3H), 1.28 (d, J=6.2 Hz, 3H), 1.45-1.91 (m, 8H), 2.96 (m, 1H), 3.42 (m, 2H), 3.56 (m, 1H), 3.64 (dd, J=2.2 Hz, J=6.7 Hz, 1H), 3.85 (m, 1H), 4.18 (m, 1H), 5.91 (bs, 1H).

¹³C NMR (300 MHz, CDCl₃): δ −4.7, −4.3, 15.6, 18.0, 19.2, 21.5, 22.8, 24.5, 25.8, 33.8, 54.0, 61.7, 63.9, 66.5, 70.6, 77.2, 168.9.

### EXAMPLE 3

### Preparation of (3S)-3-((R)-1-(tert-butyldimethylsilyloxy)ethyl)-4-((1R,2S,3S)-2-hydroxy-3-propoxycyclohexyl)azetidin-2-one (compound 2c)

Instead of using methanol as in example 1, n-propanol is used as a nucleophilic compound resulting the title compound in 69% yield. 3×2.5 mol % Yb(OTf)₃ is used as a catalyst.

m.p. 119-121° C. (from hexane).

MS, (M+H)⁺=386 m/z.

IR (KBr): 3449, 3293, 2935, 2858, 1756, 1715, 1255, 1106, 1087, 969, 837, 776 cm⁻¹.

¹H NMR (300 MHz, CDCl₃): δ 0.06 (s, 6H), 0.86 (s, 9H), 0.88 (t, J=7.4 Hz, 3H), 1.24 (d, J=6.2 Hz, 3H), 1.41-1.58 (m, 7H), 1.64 (m, 1H), 1.85 (m, 1H), 2.38 (bs, 1H), 2.91 (m, 1H), 3.30 (m, 1H), 3.40 (m, 2H), 3.62 (dd, J=1.0 Hz, J=6.1 Hz, 1H), 3.81 (m, 1H), 4.13 (m, 6.2, 1H), 6.38 (bs, 1H).

¹³C NMR (300 MHz, CDCl₃): δ −4.7, −4.3, 10.7, 18.0, 19.2, 21.2, 22.7, 23.3, 24.3, 25.8, 39.5, 54.1, 61.6, 66.4, 70.2, 70.5, 77.3, 169.2.

### EXAMPLE 4

### Preparation of (3S)-3-((R)-1-(tert-butyldimethylsilyloxy)ethyl)-4-((1R,2S,3S)-2-hydroxy-3-isopropoxycyclohexyl)azetidin-2-one (compound 2d)

Instead of using methanol as in example 1, iso-propanol is used as a nucleophilic compound resulting the title compound in 30% yield. 4×3 mol % Yb(OTf)₃ is used as a catalyst.

m.p. 121-123° C. (from hexane)

MS, (M+H)⁺=386 m/z

IR (KBr): 3480, 3253, 2932, 1755, 1715, 1256, 1141, 1060, 966, 831, 775 cm⁻¹.

¹H NMR 8300 MHz, CDCl₃): δ0.09 (s, 6H), 0.89 (s, 9H), 1.12 (d, J=6.4 Hz, 3H), 1.14 (d, J=6.6 Hz, 3H), 1.28 (d, J=6.1 Hz, 3H), 1.45-1.93 (m, 8H), 2.96 (dd, J=1.4 Hz, J=5.9 Hz, 1H); 3.48 (m, 1H), 3.61-3.72 (m, 2H), 3.77 (m, 1H), 4.18 (m, 1H), 6.04 (bs, 1H).

¹³C NMR (300 MHz, CDCl₃): δ −4.7, −4.3, 18.0, 19.3, 21.4, 22.3, 22.7, 23.0, 25.3, 25.8, 39.5, 54.0, 61.6, 66.4, 69.1, 71.2, 74.5, 169.2

### EXAMPLE 5

### Preparation of (3S)-3-((R)-1-(tert-butyldimethylsilyloxy)ethyl)-4-((1R,2S,3S)-2-hydroxy-3-butoxycyclohexyl)azetidin-2-one (compound 2e)

Instead of using methanol as in example 1, n-butanol is used as a nucleophilic compound resulting the title compound in 89% yield. 3×4 mol % Yb(OTf)₃ is used as a catalyst.

m.p. 105-106° C. (from hexane)

MS, (M+H)⁺=400 m/z

IR (KBr): 3447, 3285, 2933, 2855, 1755, 1715, 1255, 1142, 1091, 965, 836, 776 cm⁻¹.

¹H NMR (300 MHz, CDCl₃): δ 0.10 (s, 6H), 0.90 (s, 9H), 0.92 (t, J=7.2 Hz, 3H), 1.29 (d, J=6.2 Hz, 3H), 1.33-1.73 (m, 1H), 1.88 (m, 1H), 2.96 (dd, J=2.1 Hz, 6.1 Hz, 1H), 3.32-3.41 (m, 2H), 3.49 (m, 1H), 3.65 (dd, J=2.2 Hz, J=6.5 Hz, 1H), 3.85 (m, 1H), 4.18 (m, 1H), 5.92 (bs, 1H).

¹³C NMR (300 MHz, CDCl₃): δ −4.7, −4.3, 13.9, 18.0, 19.2, 21.2, 22.7, 24.3, 25.8, 32.3, 39.5, 54.1, 61.6, 66.4, 68.3, 70.5, 77.3, 169.2.

### EXAMPLE 6

### Preparation of (3S)-3-((R)-1-(tert-butyldimethylsilyloxy)ethyl)-4-((1R,2S,3S)-3-(2-fluoroethoxy)-2-hydroxycyclohexyl)azetidin-2-one (compound 2 f)

Instead of using methanol as in example 1, 2-fluoroethanol is used as a nucleophilic compound resulting the title compound in 53% yield. 2×1 mol % Yb(OTf)₃ is used as a catalyst.

m.p. 106-110° C. (from hexane)

MS, (M+H)⁺=390 m/z.

IR (KBr): 3409, 2931, 2861, 1755, 1739, 1252, 1104, 1056, 964, 837, 776 cm⁻¹.

¹H NMR (300 MHz, CDCl₃): δ 0.10 (s, 6H), 0.89 (s, 9H), 1.28 (d, J=6.2 Hz, 3H), 1.44-1.92 (m, 7H), 2.97 (m, 1H), 3.50 (m, 1H), 3.56-3.86 (m, 2H), 3.66 (dd, J=2.3 Hz, J=4.9 Hz, 1H), 3.90 (m, 1H), 4.45 (t, J=4.2 Hz, 1H), 4.61 (t, J=4.2 Hz, 1H), 5.98 (bs, 1H).

¹³C NMR (300 MHz, CDCl₃): δ −4.7, −4.3, 18.0, 19.0, 21.3, 22.7, 24.1, 25.8, 39.5, 54.0, 61.5, 66.4, 67.8 (J^{19F13C}=20 Hz), 70.1, 78.1, 83.2 (J^{19F13C}=169 Hz), 169.1.

### EXAMPLE 7

### Preparation of (3S)-3-((R)-1-(tert-butyldimethylsilyloxy)ethyl)-4-((1R,2S,3S)-3-(2-chloroethoxy)-2-hydroxycyclohexyl)azetidin-2-one (compound 2g)

Instead of using methanol as in example 1, 2-chloroethanol is used as a nucleophilic compound resulting the title compound in 47% yield. 3×3 mol % Yt(OTf)3 is used as a catalyst.

m.p. 97-100° C. (from hexane)

MS, (M+H)⁺=406 m/z.

IR (KBr): 3440, 2933, 2858, 1757, 1714, 1256, 1106, 1057, 964, 837, 776 cm⁻¹.

¹H NMR (300 MHz, CDCl₃): δ 0.07 (s, 6H), 0.87 (s, 9H), 1.25 (d, J=6.2 Hz, 3H), 1.43-1.51 (m, 5H), 1.68 (m, 1H), 1.88 (m, 1H), 2.37 (bs, 1H), 2.94 (m, 1H), 3.48 (m, 1H), 3.55-3.65 (m, 4H), 3.75 (m, 1H), 3.86 (m, 1H), 4.15 (d, J=6.2, 1H), 6.29 (bs, 1H).

¹³C NMR (300 MHz, CDCl₃): δ −4.8, −4.4, 17.9, 19.0, 21.2, 22.6, 24.3, 25.7, 39.5, 43.1, 53.9, 61.6, 66.3, 68.8, 70.1, 78.0, 169.0.

### EXAMPLE 8

### Preparation of 3-((1 S,2S,3R)-3-((3S)-3-((R)-(tert-butyldimethylsilyloxy)ethyl)-4-oxoazetidin-2-yl)-2-hydroxycyclohexyloxy)propanenitrile (compound 2h)

Instead of using methanol as in example 1, 3-hydroxypropionitrile is used as a nucleophilic compound resulting the title compound in 55% yield. 3×10 mol % Sn(OTf)₂ is used as a nucleophile.

m.p. 133-135° C. (from hexane)

MS, (M+H)⁺=397 m/z.

IR (KBr): 3508, 3153, 3088, 2935, 2260, 1756, 1708, 1250, 1138, 1102, 1053, 964, 837, 777 cm⁻¹.

¹H NMR (300 MHz, CDCl₃): δ 0.10 (s, 6H). 0.90 (s; 9H); 1.30 (d, J=6.1 Hz, 3H), 1.48-1.92 (m, 8H), 2.60 (t, J=6.2 Hz, 2H), 2.98 (dd, J=6.3 Hz, 1H), 3.53 (m, 1H), 3.62 (m, 2H), 2.78 (m, 1H), 3.90 (m, 1H), 4.18 (m, 1H), 5.91 (bs, 1H).

¹³C NMR (300 MHz, CDCl³): δ −4.7, −4.3, 18.0, 19.1, 19.3, 20.1, 22.7, 23.9, 25.8, 39.3, 54.0, 61.4, 63.3, 66.4, 70.0, 78.2, 118.0, 169.2

### EXAMPLE 9

General method for the preparation of compound of formula 2 using microwave irradiation. Experiments are performed on Biotage Initiator Sixty and Milestone Mycrosynth:

### Biotage Initiator Sixty:

Temperature: 60-250° C.
Pressure range 0-20 bar (2 MPa, 290 PSI)
Power range: 0-300 W at 2.45 GHz

### Milestone Microsynth:

Temperature: 25-250° C.
Pressure range: 0-55 bar
Power range: 0-1000 W at 2.45 GHz

### Preparation of (3S)-3-((R)-1-(tert-butyldimethylsilyloxy)ethyl)-4-((1R,2S,3S)-2-hydroxy-3-methoxycyclohexyl)azetidin-2-one (compound 2a)

To a solution of 100 mg (0.31 mmol) of (3S,4R)-3-[(1R)-1-(tert-butyldimethylsilyloxy)ethyl]-4-[(1′R,2′S, 3′R)-1′,2′-epoxycyclohexan-3′-yl]azetidin-2-one (compound 1) in methanol (1.5 ml), a portion (2.9 mg, 3 mol. %) of ytterbium (III) trifluoromethane is added. The mixture is stirred at 80° C. for one hour under microwave irradiation (Biotage Initiator Sixty), then 50 ml of water is added and the obtained solid filtered and dried to give 78 mg (HPLC: 94 area %, yield 67%) white substance of the title compound.

According to the above general method the following compounds using microwave irradiation are prepared:

### EXAMPLE 10

### Preparation of (3S)-3-((R)-1-(tert-butyldimethylsilyloxy)ethyl)-4-((1R,2S,3S)-2-hydroxy-3-ethoxycyclohexyl)azetidin-2-one (compound 2b)

Instead of using methanol as in example 9, ethanol is used as a nucleophilic compound resulting the title compound in 67% yield. 3 mol % Yb(OTf)₃ is used as a catalyst.

### EXAMPLE 11

### Preparation of (3S)-3-((R)-1-(tert-butyldimethylsilyloxy)ethyl)-4-((1R,2S,3S)-2-hydroxy-3-propoxycyclohexyl)azetidin-2-one (compound 2c)

Instead of using methanol as in example 9, n-propanol is used as a nucleophylic compound resulting the title compound in 59% yield. 3 mol % Yb(OTf)₃ is used as a catalyst.

### EXAMPLE 12

### Preparation of (3S)-3-((R)-1-(tert-butyldimethylsilyloxy)ethyl)-4-((1R,2S,3S)-2-hydroxy-3-isopropoxycyclohexyl)azetidin-2-one (compound 2d)

Instead of using methanol as in example 9, iso-propanol is used as a nucleophilic compound resulting the title compound in 71% yield. 3 mol % Yb(OTf)₃ is used as a catalyst.

### EXAMPLE 13

### Preparation of (3S)-3-((R)-1-(tert-butyldimethylsilyloxy)ethyl)-4-((1R,2S,3S)-2-hydroxy-3-butoxycyclohexyl)azetidin-2-one (compound 2e)

Instead of using methanol as in example 9, n-butanol is used as a nucleophilic compound resulting the title compound in 75% yield. 3 mol % Yb(OTf)₃ is used as a catalyst.

### EXAMPLE 14

### Preparation of (3S)-3-((R)-1-(tert-butyldimethylsilyloxy)ethyl)-4-((1R,2S,3S)-3-(2-fluoroethoxy)-2-hydroxycyclohexyl)azetidin-2-one (compound 2f)

Instead of using methanol as in example 9 but without using a catalyst, 2-fluoroethanol is used as a nucleophilic compound resulting in 53% yield. The reaction is carried out at 60° C. for 1.5 minutes under microwave irradiation (Biotage Initiator Sixty).

### EXAMPLE 15

### Preparation of (3S)-3-((R)-1-(tert-butyldimethylsilyloxy)ethyl)-4-((1R,2S,3S)-3-(2-chloroethoxy)-2-hydroxycyclohexyl)azetidin-2-one (compound 2g)

Instead of using methanol as in example 9, 2-chloroethanol is used as a nucleophilic compound resulting the title compound in 47% yield. 3 mol % Yt(OTf)₃ is used as a catalyst.

### EXAMPLE 16

### Preparation of (3S)-3-((R)-1-(tert-butyldimethylsilyloxy)ethyl)-4-(1R,2S,3S)-3-iso-butoxy-2-hydroxycyclohexyl)azetidin-2-one (compound 2i)

Instead of using methanol as in example 9, iso-butanol is used as a nucleophilic compound resulting the title compound in 71% yield. 3 mol % Yb(OTf)₃ is used as a catalyst.

¹H NMR (300 MHz, CDCl₃): δ 0.10 (s, 6H), 0.89-0.96 (m, 15H), 1.29 (d, J=6.21, 3H), 1.44-1.83 (m, 8H), 2.96 (m, 1H), 2.80-2.92 (m, 1H), 3.09-3.30 (m, 1H), 3.38 (m, 1H), 3.65 (dd, J=6.49, 2.15, 1H), 3.86 (m, 1H), 4.18 (m, 1H), 5.93 (bs, 1H)

### EXAMPLE 17

### Preparation of (3S,4R)-3-[(1R)-1-(tert-butyldimethylsilyloxy)ethyl]-4-[(1S,2S,6R)-1-hydroxy-2-methylaminocyclohexyl-6-yl]azetidin-2-one (compound 2j)

A solution of 100 mg (0.31 mmol) of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound 1) in 33 wt % of methylamine in ethanol (3.0 ml) is stirred at 100° C. for 30 minutes under microwave irradiation (Biotage Initiator Sixty), then reaction mixture is concentrated in vacuo. The obtained residue is dissolved in ethyl acetate and the resulted solution washed with water and dried over MgSO₄ and then evaporated to give 59 mg (54%) of the title compound.

¹H NMR (300 MHz, CDCl₃): δ 0.12 (s, 6H), 0.91 (s, 9H), 1.31 (d, J=6.3, 3H), 1.40-1.70 (m, 5H), 2.00 (m, 2H), 2.44 (s, 3H), 2.55 (m, 1H), 3.06 (d, J=6.9, 1H), 3.65 (dd, 1H), 3.71 (dd, 1H), 4.20 (m, 1H), 5.99 (bs, 1H).

The following table summarized obtained yields of the processes according to the above described examples:

| | | Yield (%) | Yield (%) |
|---|---|---|---|
| | | obtained by | obtained by |
| Compound of | | ultrasonic | microwave |
| general formula 2 | R | irradiation | irradiation |
| a | —OMe | 84 | 67 |
| b | —OEt | 75 | 67 |
| c | —O-nPr | 69 | 59 |
| d | —O-isoPr | 30 | 71 |
| e | —O-nBu | 89 | 75 |
| f | —OCH2CH2F | 53 | 53 |
| g | —OCH2CH2Cl | 47 | 47 |
| h | —OCH2CH2CN | 55 | |
| i | —O-isoBu | | 71 |
| j | —NHMe | | 54 |

### EXAMPLE 18

### Preparation of (3S)-3-((R)-1-(tert-butyldimethylsilyloxy)ethyl)-4-((1R,2R,3S)-3-chloro-2-hydroxycyclohexyl)azetidin-2-one (compound 3)

To a solution of 2.67 g (8.20 mmol) of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound 1) in ethanol (16 ml) 1.76 of ammonium chloride solution is added. Obtained mixture is warmed and stirred at 130° C. for three hours under microwave irradiation (Milestone Microsynth), then the reaction mixture is concentrated in vacuo and the obtained residue is dissolved in ethyl acetate. The resulted solution is washed with saturated sodium hydrogenecarbonate solution, dried over MgSO₄, the organic solvent is evaporated and the obtained dry substance is purified by flash chromatography eluting with a 2:1 mixture of hexane and ethyl acetate as mobile phase. After evaporating of the organic solvent 2.03 g (HPLC: 92 area %, 66% yield) of the title compound is obtained.

m.p.=140-160° C.

IR (KBr): 427, 2948, 2857, 1760, 1723, 1257, 1140 cm⁻¹.

MS, (M+H)⁺=362 m/z.

¹H NMR (300 MHz, CDCl₃): δ 0.08 (s, 3H), 0.09 (s, 3H), 0.88 (s, 9H), 1.27 (d, J=6.2, 3H), 1.49-1.82 (m, 5H), 2.11 (m, 2H), 2.74 (bs, 1H),2.96 (dd, J=1.7, 6.2, 1H), 3.63 (dd, J=2.0, 5.8, 1H), 3.93 (t, J=3.0, 1H), 4.15 (m, 2H), 6.30 (s, 1H).

¹³C NMR (300 MHz, CDCl₃): δ −4.7, −4.3, 18.0, 19.0, 21.1, 22.7, 25.8 (3), 28.0, 38.2, 54.1, 60.4, 61.6, 66.5, 72.3, 169.0

### EXAMPLE 19

### Preparation of (3S)-3-((R)-1-(tert-butyldimethylsilyloxy)ethyl)-4-((1R,2S,3S)-3-chloro-2-hydroxycyclohexyl)azetidin-2-one (compound 3)

To a solution of 327 mg (1.0 mmol) of (3S,4R)-3-[(1R)-1-(tert-butyldimethylsilyloxy)ethyl]-4-[(1′R,2′S,3′R)-1′,2′-epoxycyclohexan-3′-yl]azetidin-2-one (compound 1) in ethanol (4 ml), a solution of 108 mg of ammonium chloride in 0.7 ml water is added and heated at reflux temperature for 24 hours. After workup which is identical as in example 18, 16% of the title compound is recovered, with identical spectroscopic properties as in example 18.

1. A process for the preparation of 1′-hydroxy-2′-substituted cyclohexyl azetidin-2-one compound of the formula 2 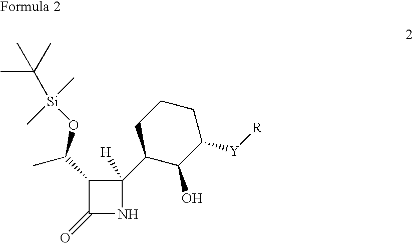
wherein Y is O, S or NH; R is hydrogen, or selected from a) phenyl group optionally substituted by one or two substituents each selected from nitro, halo, C₁-C₄ alkyl, C₁-C₄ alkoxy, hydroxy, trifluoromethyl, and cyano group, b) a 1- or 2-naphthyl group, and c) oxyranyl, aziridinyl, pirolyl, imidazolyl, pyrazolyl, furanyl, thiophenyl, thiazolyl, isothiazolyl, triazolyl, tetrazolyl, oxazolyl, isooxazolyl, azepinyl, pyridinyl, pyrimidinyl, pyrazinyl, pyridazinyl, triazinyl, pyranyl and their hydrogenated analogs, unsubstituted or optionally substituted by one or two substituents each selected from nitro, nitroso, amino, imino, sulfonyl, chlorosulfonyl, halo, C₁-C₄ alkyl, C₁-C₄ alkoxy, C₁-C₄ alkoxycarbonyl, C₁-C₄ alkylthio, C₁-C₄ alkylthiocarbonyl, hydroxy, trifluoromethyl, carboxyaldehyde or cyano group; or R₁(CHₘ)ₙ— group where n is an integer from 1 to 11, m is an integer from 1 to 2 and R₁ is a) hydrogen, halo, hydroxy, cyano, C₁-C₄ alkoxy, C₁-C₄ alkoxycarbonyl, nitro, nitroso, imino, sulfonyl, chlorosulfonyl, C₁-C₄ alkylthio, C₁-C₄ alkylthiocarbonyl, or b) C₁-C₆ alkyl, C₂-C₆ alkenyl, C₃-C₇ cycloalkyl, optionally substituted by one or two substituents each selected from halo, hydroxy, ciano, carboxyaldehyde, C₁-C₄ alkoxy, C₁-C₄ alkoxycarbonyl, nitro, nitroso, amino, imino, sulfonyl, chlorosulfonyl, C₁-C₄ alkylthio, C₁-C₄ alkylthiocarbonyl group,
which comprises reacting (3S,4R)-3-[(1R)-1-(tert-butyldimethylsilyloxy)ethyl]-4-[(1′R,2′S,3′R)-1′,2′-epoxycyclohexan-3′-yl]azetidin-2-one of formula 1, 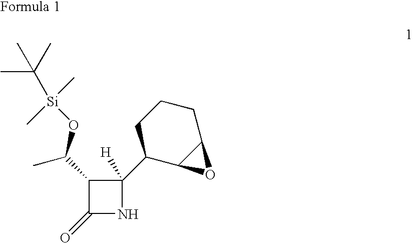
with the nucleophile compound of the formula 4
RYH Formula 4
wherein Y and R are as defined above,
which nucleophile if in a liquid form can act as a solvent itself, in a suitable solvent and in the presence of a suitable catalyst selected from the group of salt of trifluoromethanesulfonic acid under a) ultrasonic irradiation or b) under microwave irradiation of reaction mixture and after completing the reaction the title compound is isolated and purified.

2. The process according to claim 1, wherein the solvent comprise water, C₁-C₆ alcohol, N,N-dimethylformamide, N,N-dimethylacetamide, acetonitrile, tetrahydrofuran, dioxane, 1-methylpyrrolidinone, 1,3-dimethyl-3,4,5,6-tetrahydro-2(1H)-pyrimidone, dichloromethane, dimethylsulfoxide, C₁-C₄ alkyl acetate or aromatic hydrocarbon, or mixtures thereof.

3. The process according to claim 1, wherein the solvent comprise methanol, ethanol, n-propanol, iso-propanol, n-butanol, or sec-butanol.

4. The process according to claim 1, wherein the catalyst comprise ytterbium (III) triflate, stannous (II) triflate and dysprosium (III) triflate.

5. The process according to claim 1, wherein ultrasonic irradiation is in the frequency range from 20 to 80 kHz.

6. The process according to claim 1, wherein ultrasonic irradiation is in the frequency range from 30 to 50 kHz.

7. A process for the preparation of 1′-hydroxy-2′-substituted cyclohexyl azetidin-2-one compound of the formula 3 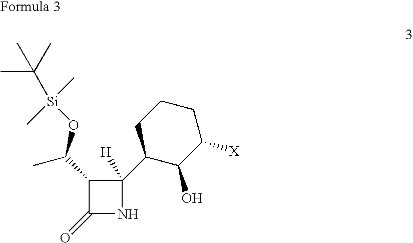
wherein X is halogen atom, thiocyanate, isothiocyanate, hydroxy, sulphate, nitro, sulfide, sulfite, carbonate or acetate group, which comprises reacting (3S,4R)-3-[(1R)-1-(tert-butyldimethylsilyloxy)ethyl]4-[(1′R,2′S,3′R)-1′,2′-epoxycyclohexan-3′-yl]azetidin-2-one of the formula 1, 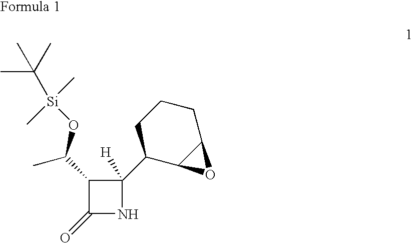
with the compound of formula 5
NH₄X Formula 5
wherein X is as defined above, in a suitable solvent under a) microwave irradiation or, b) without microwave irradiation of the reaction mixture and after completing the reaction the title compound is isolated and purified.

8. The process according to claim 7, wherein the solvent comprise water, C₁-C₆ alcohol, N,N-dimethylformamide, N,N-dimethylacetamide, acetonitrile, tetrahydrofuran, dioxan, 1-methylpyrrolidinone, 1,3-dimethyl-3,4,5,6-tetrahydro-2-(1H)-pyrimidone, dichloromethane, chloroform, dimethylsulfoxide, ethyl acetate.

9. A, process according to claim 1, which comprise the use of the compound of the formula 6 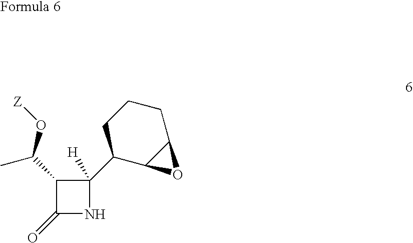
as starting compound instead of the compound of formula 1, wherein Z is any suitable hydroxy protecting group.
